(19) United States
(12) Patent Application Publication
(10) Pub. No.: US 2024/0262768 A1
(43) Pub. Date: Aug. 8, 2024

(54) METHOD FOR PRODUCING ISOPROPYL ALCOHOL

(71) Applicant: NIPPON SHOKUBAI CO., LTD., Osaka-shi, Osaka (JP)

(72) Inventor: Junji OKAMURA, Suita-shi, Osaka (JP)

(21) Appl. No.: 18/560,770

(22) PCT Filed: May 18, 2022
(86) PCT No.: PCT/JP2022/020634

§ 371 Date: Nov. 14, 2023

(30) Foreign Application Priority Data
May 19, 2021 (JP) 2021-084791

(51) Int. Cl. C07C 29/145; B01J 23/42; B01J 23/46; B01J 23/63; B01J 23/745; B01J 23/755; B01J 23/80; B01J 37/00; B01J 37/02; B01J 37/03; B01J 37/04; B01J 37/08; C01B 3/32

(52) U.S. Cl. CPC C07C 29/145; B01J 23/42; B01J 23/462; B01J 23/63; B01J 23/745; B01J 23/755; B01J 23/80; B01J 37/009; B01J 37/0236; B01J 37/031; B01J 37/04; B01J 37/08; C01B 3/326

(57) ABSTRACT

The present invention aims to provide a method for producing isopropyl alcohol applicable to production of carbon-neutral isopropyl alcohol and capable of efficiently producing isopropyl alcohol with simple operations. The present invention relates to a method for producing isopropyl alcohol, the method including: a step (1) of reacting ethanol and water in the presence of a catalyst to obtain acetone; a step (2) of purifying the acetone; and a step (3) of reducing the acetone to obtain isopropyl alcohol.

### TECHNICAL FIELD

The present invention relates to methods for producing isopropyl alcohol.

### BACKGROUND ART

Isopropyl alcohol is a key chemical widely used as a solvent, diluent, or the like in various industrial applications.

Conventionally, isopropyl alcohol has been produced mainly by a hydration reaction of propylene that is obtained by thermal decomposition of petroleum (Patent Literature 1), or by hydrogenation of acetone which is a by-product of production of phenol from benzene and propylene (Patent Literature 2).

However, amid recent global efforts to build a decarbonized society, there has been a strong desire to replace conventional fossil resource-derived isopropyl alcohol with carbon-neutral isopropyl alcohol.

A proposed method for producing carbon-neutral isopropyl alcohol is a method for producing isopropyl alcohol from plant-derived materials using an isopropyl alcohol-producing bacterium (Patent Literature 3). The method proposed in Patent Literature 3 provides carbon-neutral isopropyl alcohol in which all the carbons are derived from plant-derived materials such as glucose.

However, production of isopropyl alcohol using isopropyl alcohol-producing bacteria requires several tens of hours of reaction time, and the accumulated concentration of isopropyl alcohol is insufficient, resulting in unsatisfactory production efficiency. In addition, the production of isopropyl alcohol requires complicated operation and additional reagents. For example, in order to control the pH of the culture solution within a predetermined range, it is essential to add a pH adjuster such as an aqueous ammonia solution or a NaOH aqueous solution.

### CITATION LIST

### Patent Literature

Patent Literature 1: JP H08-291092
Patent Literature 2: JP H02-270829
Patent Literature 3: WO 2009/008377

### SUMMARY OF INVENTION

### Technical Problem

As described above, conventional production methods are to provide fossil resource-derived isopropyl alcohol and are not capable of providing carbon-neutral isopropyl alcohol, which is considered essential for building a decarbonized society. Production of isopropyl alcohol using isopropyl alcohol-producing bacteria can produce carbon-neutral isopropyl alcohol, but has issues in terms of industrial production, such as slow isopropyl alcohol production rate and need for operations such as pH adjustment.

The present invention has been made in view of these circumstances and aims to provide a method for producing isopropyl alcohol applicable to production of carbon-neutral isopropyl alcohol and capable of efficiently producing isopropyl alcohol with a simple operation.

### Solution to Problem

The present inventors conducted extensive studies to achieve the aforementioned object and successfully completed the present invention.

That is, the present invention relates to a method for producing isopropyl alcohol, the method including:
a step (1) of reacting ethanol and water in the presence of a catalyst to obtain acetone;
a step (2) of purifying the acetone; and
a step (3) of reducing the acetone to obtain isopropyl alcohol.

Preferably, in the production method, a reducing agent used in the step (3) includes hydrogen obtained in the step (1).

Preferably, in the production method, the reducing agent used in the step (3) includes hydrogen obtained in the step (2).

Preferably, in the production method, a percentage of an amount of carbon dioxide in all components introduced in the step (3) is less than 10 mol %.

Preferably, in the production method, the ethanol is derived from biomass as a raw material.

### Advantageous Effects of Invention

According to the present invention, carbon-neutral isopropyl alcohol can be produced efficiently with a simple operation.

### BRIEF DESCRIPTION OF DRAWINGS

FIG. 1 is an exemplary preferred embodiment of a production process of isopropyl alcohol in the present disclosure.

FIG. 2 is another exemplary preferred embodiment of the production process of isopropyl alcohol in the present disclosure.

FIG. 3 is another exemplary preferred embodiment of the production process of isopropyl alcohol in the present disclosure.

### DESCRIPTION OF EMBODIMENTS

The present disclosure will be described in detail below. A combination of two or more of individual preferred embodiments of the present invention described below is also a preferred embodiment of the present invention.

### (Method for Producing Isopropyl Alcohol of the Present Disclosure)

### <Step (1)>

A method for producing isopropyl alcohol (sometimes also referred to as isopropanol) of the present disclosure includes reacting ethanol and water in the presence of a catalyst to obtain acetone (hereinafter referred to as a step (1)).

### (Catalyst)

A catalyst used in the step (1) (also referred to as an acetone synthesis catalyst) should be any one containing a metal element. The catalyst is preferably one containing one or more elements selected from alkali metals, alkaline earth metals, iron, manganese, zinc, copper, aluminum, and zirconium.

The metal element may be present in any state in the catalyst used in the step (1). For example, the metal element may be contained in a metal oxide, the metal element may be contained in a support, or the metal element or a compound of the metal element may be supported on a support. A metal oxide containing the metal element may be supported on a support. The metal oxide may be a complex metal oxide.

Examples of the complex metal oxide include spinel-type, perovskite-type, magnetoplumbite-type, and garnet-type oxides, with a spinel-type oxide being preferred.

The catalyst used in the step (1) is preferably one containing iron from the viewpoint of catalytic activity. More preferably, the catalyst is one containing iron (Fe) and one or more metals (Me) selected from the group consisting of magnesium (Mg), calcium (Ca), manganese (Mn), and zinc (Zn).

The catalyst containing iron (Fe) and one or more metals (Me) selected from the group consisting of magnesium (Mg), calcium (Ca), manganese (Mn), and zinc (Zn) is preferably a complex iron oxide (sometimes referred to as ferrite) represented by the following formula (1):

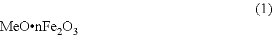

wherein Me represents one or more metals selected from the group consisting of Mg, Ca, Mn, and Zn, and n represents 1 to 6.

Specific examples of the complex iron oxide include MgO·Fe₂O₃ and ZnO·Fe₂O₃.

In the catalyst containing iron (Fe) and one or more metals (Me) selected from the group consisting of magnesium (Mg), calcium (Ca), manganese (Mn), and zinc (Zn), the amount of one or more metals (Me) selected from the group consisting of magnesium, calcium, manganese, and zinc per mole of iron is preferably 0.4 to 0.7 mol, more preferably 0.4 to 0.6 mol, still more preferably 0.45 to 0.55 mol.

In other words, n in the formula (1) is preferably 1.43 to 2.5, more preferably 1.67 to 2.5, still more preferably 2 to 2.22. Within the above range, good catalytic activity can be obtained.

When the catalyst used in the step (1) is one in which a metal element or metal oxide is supported on a support, the support may be activated carbon, silica, alumina, silica-alumina, zeolite, silica-calcia, ceria, magnesia, diatomaceous earth, or the like.

When the catalyst used in the step (1) is one in which a metal element or metal oxide is supported on a support, the mass percentage of the support is preferably 20 to 70% by mass based on 100% by mass of the entire catalyst. With such a percentage, the catalytic component can be effectively distributed on a support, and whereby the catalyst can exhibit high activity. The mass percentage of the support is more preferably 25 to 67% by mass, still more preferably 30 to 60% by mass, based on 100% by mass of the entire catalyst.

In a preferred embodiment of the present invention, the catalyst used in the step (1) is one containing aluminum (Al) as a metal element.

When the catalyst containing aluminum is a compound containing only aluminum as a metal element, the catalyst may be aluminum oxide, for example. When the catalyst containing aluminum is a complex metal oxide containing other metal element(s), the catalyst may be a complex metal oxide containing aluminum and Sn, Pb, Zn, Fe, In, or the like. When the catalyst used in the step (1) contains aluminum in a support, the support may be alumina (Al₂O₃), silica-alumina, or the like. From the viewpoint of catalyst performance, the support is more preferably Al₂O₃.

When the catalyst used in the step (1) contains iron and aluminum, the amount of aluminum per mole of iron is preferably 0.01 to 0.5 mol, more preferably 0.05 to 0.5 mol, still more preferably 0.1 to 0.4 mol. Within the above range, the catalyst can exhibit good durability.

In a preferred embodiment of the present invention, the catalyst used in the step (1) is one containing zirconium (Zr) as a metal element.

When the catalyst containing zirconium is a compound containing only zirconium as a metal element, the catalyst may be zirconium oxide, for example.

When the catalyst used in the step (1) contains iron and zirconium, the amount of zirconium per mole of iron is preferably 0.2 to 2.0 mol, more preferably 0.3 to 1.8 mol, still more preferably 0.4 to 1.5 mol. Within the above range, the catalyst can exhibit good durability.

In the catalyst used in the step (1), the total amount of iron, aluminum, zirconium, and one or more metals (Me) selected from the group consisting of magnesium, calcium, manganese, and zinc is preferably 50 to 100% by mass, more preferably 80 to 100% by mass, based on 100% by mass of the catalyst.

The catalyst used in the step (1) may be produced by any method, and can be produced by an impregnation method, a precipitation method, or a coprecipitation method, for example. More preferred is a coprecipitation method. A coprecipitation method is capable of providing a coprecipitate (sometimes referred to as a catalyst precursor) in which metal element(s) that is to be a component constituting a catalyst is uniformly and highly distributed. As a result, a catalyst with excellent performance can be produced.

In the coprecipitation method, a catalyst can be produced by mixing an aqueous solution of a compound of a metal element that is to be contained in a catalyst, and adding a basic aqueous solution to the solution containing a metal element compound to precipitate a poorly soluble salt at the same time.

The metal element compound may be any one soluble in water, and may be selected from compounds such as chlorides, hydrochlorides, sulfates, and nitrates depending on the type of metal element.

Non-limiting examples of the alkali added to precipitate a poorly soluble salt of a metal element include sodium hydroxide, an aqueous ammonia solution, and potassium hydroxide.

The coprecipitation method may further include, in addition to the adding a basic aqueous solution to the solution containing a metal element compound to obtain a coprecipitate, filtering the coprecipitate, drying the filtered coprecipitate, and firing the dried coprecipitate.

When the catalyst is produced by the coprecipitation method, the amount of metal element(s) in the solution may be varied as appropriate.

In production of a catalyst in which a metal element is supported on a support, an impregnation method can be used.

The impregnation method includes mixing a solution of a metal element compound and a support and drying the mixture. Thereby, a catalyst in which the metal element is supported on the support can be produced.

The metal element compound may be any one soluble in solvents such as water, and may be selected from compounds such as chlorides, hydrochlorides, sulfates, and nitrates depending on the type of the metal element.

The impregnation method may include mixing a solution of a metal element compound and a support, drying the mixture, and firing the dried mixture.

When the catalyst is produced by the impregnation method, the amount of metal element(s) in the solution may be varied as appropriate.

The state of distribution of each catalytic component in the produced catalyst can be evaluated using, for example, an electron microprobe analyzer (EPMA). When EPMA is used, the distribution of a metal component (e.g., aluminum) in the catalyst can be evaluated as follows: the X-ray dose is measured in a plane region having 900 μm sides in the X-axis and Y-axis directions in the catalyst surface; the average value (S) and standard deviation (σ) are calculated from the X-ray dose at arbitrary points in the region; and the ratio (σ/S) of standard deviation (σ) to average value (S) is determined. Thereby, the distribution of aluminum in the catalyst can be evaluated.

The value of the ratio (σ/S) is preferably less than 0.25, more preferably less than 0.2, still more preferably less than 0.15.

### (Reaction Between Ethanol and Water)

In the step (1) in the method for producing isopropyl alcohol of the present invention, ethanol and water as raw materials are brought into contact with a catalyst. Thereby, a reaction product containing acetone, hydrogen, and carbon dioxide can be obtained.

Before bringing ethanol and water as raw materials into contact with the catalyst in the step (1), components adhering to the catalyst may be removed. This allows the catalyst to function more sufficiently. The components adhering to the catalyst may be removed by any method. The method may be flowing an inert gas through the catalyst under heating, for example.

The reaction of the step (1) is not limited, and may be either batchwise or continuous. From the viewpoint of productivity, preferably, the reaction of the step (1) is continuous.

The reaction of the step (1) is preferably a gas phase reaction. The type of the reaction by a gas phase reaction may be a fixed-bed, moving-bed, or fluidized-bed type. Preferred is a fixed-bed type, which is simpler.

When the reaction of the step (1) is of a fixed-bed type, a mixture of gaseous ethanol and gaseous water (sometimes referred to as steam) may be supplied as a feed gas to the reactor and brought into contact with the catalyst. Alternatively, gaseous ethanol and steam may be separately supplied to the reactor as feed gases and brought into contact with the catalyst.

Gaseous ethanol can be obtained, for example, by heating liquid ethanol in a vaporizer. Gaseous water can be obtained, for example, by heating water in a vaporizer.

The feed gas may contain an inert gas such as nitrogen or helium. Here, the feed gas includes all gases supplied to the reactor.

The concentration of ethanol in the feed gas is preferably 3 to 66 mol %. With such a percentage, isopropyl alcohol can be produced with high productivity. The concentration of ethanol in the feed gas is more preferably 5 to 50 mol %.

The molar ratio of water to ethanol in the feed gas is preferably 0.5 to 10. With such a ratio, the reaction between ethanol and water is performed more efficiently. The molar ratio of water to ethanol in the feed gas is more preferably 1 to 5.

The ethanol used for the feed gas is not limited, and may be one obtained by any method. Examples of the ethanol include ethanol obtained by hydration reaction of ethylene and bioethanol produced from biomass materials such as carbohydrates (e.g., sugarcane), starches (e.g., grains), and celluloses (e.g., plants). The ethanol used for the feed gas preferably contains bioethanol.

The amount of bioethanol in 100% by mass of ethanol is preferably 50% by mass or more, more preferably 75% by mass or more, still more preferably 90% by mass or more.

The reaction of the step (1) can be performed under reduced pressure, normal pressure, or increased pressure. The reaction pressure is preferably 0.07 MPa to 0.2 MPa, more preferably 0.1 MPa to 0.15 MPa.

The reaction temperature in the step (1) is preferably 250° C. to 600° C., more preferably 300° C. to 550° C., still more preferably 330° C. to 500° C.

When the reaction of the step (1) is a gas phase reaction, the feed gas preferably has a space velocity of 300 to 10,000 (l/h), more preferably 400 to 8,000 (l/h), still more preferably 500 to 6,000 (l/h).

### <Step (2)>

The method for producing isopropyl alcohol of the present disclosure includes a step including separating acetone from a mixture containing acetone (hereinafter also referred to as an acetone-containing mixture) and/or purifying the acetone (hereinafter referred to as a step (2)).

The acetone-containing mixture to be used in the step (2) contains at least the acetone obtained in the step (1). The acetone-containing mixture to be used in the step (2) may be the product obtained in the step (1) itself, or a product obtained by subjecting the product obtained in the step (1) to the step (3). The acetone-containing mixture may contain both of these.

In other words, in the method for producing isopropyl alcohol of the present disclosure, the step (2) may be performed between the step (1) and the step (3), may be performed after performing the step (1) and then the step (3), or may be performed both before and after the step (3).

The proportion of the acetone-containing mixture obtained in the step (1) in the acetone-containing mixture to be used in the step (2) is not limited. For example, it is preferably 25% by mass or more, more preferably 50% by mass or more, still more preferably 80% by mass or more, based on 100% by mass of the acetone-containing mixture to be used in the step (2).

When the acetone-containing mixture to be used in the step (2) contains a gas, the gas mainly containing hydrogen, carbon dioxide, and the like may be separated from a liquid mixture mainly containing acetone by a known gas-liquid separation method (sometimes referred to as gas-liquid separation). Here, the gas refers to a substance that exists in a gaseous state under pressurized and cooled conditions in the gas-liquid separation operation.

In the gas-liquid separation operation in the step (2), the pressure is preferably 0.1 MPa to 2 MPa, more preferably 0.2 MPa to 1 MPa.

In the gas-liquid separation operation in the step (2), the temperature is preferably 0° C. to 50° C., more preferably 5° C. to 40° C.

In the step (2), acetone may be absorbed from the gas mainly containing hydrogen, carbon dioxide, and the like. The absorption of acetone may be performed by any method. Specifically, the gas may be introduced into an absorption column, acetone in the gas is absorbed by an absorption liquid supplied from the top of the column, and the absorption liquid is collected as an acetone-containing liquid from the bottom of the column. The absorption liquid supplied from the top of the column may be any one that can effectively absorb acetone. In particular, water is preferred. The acetone-containing absorption liquid collected from the bottom of the absorption column may be combined with the liquid mixture mainly containing acetone obtained by gas-liquid separation. Thereby, the collection rate of acetone can be improved.

In the step (2), the acetone-containing mixture, which is the liquid mixture mainly containing acetone, is distilled, and whereby purified acetone can be obtained. The distillation can be performed by a known method. Examples of a known distillation method include thin-film distillation and rectification. The distillation may be continuous or batchwise.

In the step (2), only gas-liquid separation may be performed, gas-liquid separation and distillation may be performed, or only distillation may be performed. Preferably, the step (2) includes gas-liquid separation and distillation, which are performed in this order. Thereby, more sufficiently purified acetone (sometimes referred to as purified acetone) can be obtained. In this case, the purified acetone can be obtained as a distillate obtained by distillation, while a liquid mainly containing water can be obtained as a bottom liquid.

The step (2) may be performed only once or twice or more.

The purified acetone obtained in the step (2) can be used as a material to be introduced into the step (3), which is described below. As described above, the step (2) may be performed between the step (1) and the step (3), or may be performed after performing the step (1) and then the step (3). When the step (2) is performed after the step (3), the purified acetone obtained in the step (2) may be returned to the reactor for the step (3) and used as a raw material for the step (3).

The amount of acetone in the purified acetone obtained in the step (2) is preferably 90% by mass or more, more preferably 95% by mass or more, still more preferably 98% by mass or more, based on 100% by mass of the purified acetone. Such high-purity acetone with an amount of acetone within the above range, as a raw material, is subjected to acetone reduction reaction of the below-described step (3) to obtain a product. The product is subjected to gas-liquid separation to separate isopropyl alcohol and a gas in the product from each other, and whereby high-purity isopropyl alcohol can be easily obtained.

### <Step (3)>

The method for producing isopropyl alcohol of the present disclosure includes reducing acetone to obtain isopropyl alcohol (hereinafter referred to as a step (3)).

At least a portion of the acetone used in the step (3) is the acetone obtained in the step (1). When the acetone obtained in the step (1) is used in the step (3), the product obtained in the step (1) (acetone-containing mixture) may be used as is. Preferably, the purified acetone obtained in the step (2) may be used. When such high-purity acetone obtained by purification is used as a raw material for the acetone reduction reaction, high-purity isopropyl alcohol can be easily obtained by separating isopropyl alcohol and a gas in the product obtained in the step (3) from each other by gas-liquid separation.

Thus, in a preferred embodiment of the method for producing isopropyl alcohol of the present disclosure, the step (2) is performed before the step (3).

In the step (3), a substance for the reduction (sometimes referred to as a reducing agent) may be hydrogen, lithium aluminum hydride, sodium borohydride, or the like. Hydrogen is preferred.

Hydrogen used in the step (3) as a reducing agent is not limited, and may be industrially produced hydrogen. Preferably, a portion of or the entire hydrogen used in the step (3) may be the hydrogen obtained in the step (1). Alternatively, the product obtained in the step (1) may be used as is for hydrogenation.

The hydrogen used for the reduction of the step (3) may contain hydrogen separated from the acetone-containing mixture in the step (2). In the step (3), high-purity isopropyl alcohol can be easily obtained by bringing, as a reducing agent, the hydrogen separated from the acetone-containing mixture in the step (2) into contact with purified acetone.

The amount of carbon dioxide in all the components introduced into the step (3) is preferably small. The percentage of carbon dioxide in all the components introduced into the step (3) is preferably less than 10 mol %, more preferably less than 5 mol %, still more preferably less than 2 mol %. Within the above range, the catalytic activity of the catalyst used in the step (3) tends to be improved.

Thus, in a preferred embodiment of the method for producing isopropyl alcohol of the present disclosure, the step (2) is performed before the step (3) to separate carbon dioxide from the acetone-containing mixture obtained in the step (1), the resulting product is introduced into a reactor for performing the step (3), and the step (3) is then performed.

The catalyst used in the step (3) may be any catalyst such as Raney catalyst. Examples of other catalysts include solid catalysts each containing a metal element(s) such as Ba, Co, Cr, Cu, Fe, Mn, Ni, Pd, Pt, Zn, Zr, Ru, or Rh. Preferred among these is a solid catalyst containing at least one or more metal elements selected from the group consisting of Pt, Ru, Ni, Fe, and Co, with at least one or more solid catalysts selected from the group consisting of a Ru catalyst, a Ni—Pt catalyst, a Ru—Pt catalyst, and a Ni—Ru catalyst being more preferred. Use of any of these solid catalysts each containing a metal element(s) can prevent the activity inhibition caused by carbon dioxide in the acetone reduction reaction using hydrogen in the step (3). Thereby, acetone hydrogenation efficiently proceeds to lead to production of isopropyl alcohol.

The catalyst may be in the form of a metal element, a metal alloy, a metal oxide, or the like. The catalyst may also be a mixture of metal elements, a mixture of a metal element and a metal oxide, a mixture of metal oxides, or a complex metal oxide.

The catalyst may be one in which a metal element is supported on a support such as activated carbon, silica (SiO₂), alumina (Al₂O₃), titania (TiO₂), zirconia (ZrO₂), ceria (CeO₂), magnesia (MgO), or diatomaceous earth. Preferred among these are silica (SiO₂) and zirconia (ZrO₂).

These catalysts may have any shape such as a ring shape or a spherical shape.

In the step (3), one of these catalysts may be used alone or two or more of these may be used.

The catalyst used in the step (3) may be a known catalyst that reduces acetone to produce isopropyl alcohol.

The acetone-containing material to be introduced into the step (3) may be liquid or gas.

In the step (3), a reaction solvent may be used. Examples the reaction solvent include alcohols, ethers, and hydrocarbons. Water may also be used.

The reaction of the step (3) is not limited and may be either batchwise or continuous. From the viewpoint of productivity, preferably, the reaction of the step (3) is continuous.

The reaction of the step (3) is preferably a gas phase reaction. The type of the reactor by a gas phase reaction is not limited, and may be a fixed-bed or fluidized-bed type. Preferred is a fixed-bed type, which is simpler.

The reaction pressure in the reaction of the step (3) is preferably 0.1 MPa to 2 MPa, more preferably 0.1 MPa to 1 MPa.

The reaction temperature in the reaction of the step (3) is preferably 20° C. to 200° C., more preferably 25° C. to 150° C. At low reaction temperatures, which are advantageous in terms of equilibrium, hydrogenation is difficult to proceed. On the other hand, at high reaction temperatures, the hydrogenation conversion rate of acetone does not increase due to equilibrium constraints, and hydrogenolysis of acetone or isopropyl alcohol additionally occurs. Thereby, the yield tends to decrease.

When the reaction of the step (3) is a gas phase reaction, the acetone-containing material to be introduced into the step (3) preferably has a space velocity of 200 to 50,000 (l/h), more preferably 1,000 to 20,000 (l/h), still more preferably 2,000 to 10,000 (l/h).

### <Other Steps>

### (Isopropyl Alcohol Collecting Step)

The method for producing isopropyl alcohol of the present disclosure includes an isopropyl alcohol collecting step.

The isopropyl alcohol to be subjected to the collecting step is derived from the product obtained in the step (3). The product obtained by the step (3) may be used as is in the collecting step, or a product obtained by subjecting the product obtained in the step (3) to the step (2) for separation of acetone may be used in the collecting step. Also, both of these products may be used in the collecting step.

When the isopropyl alcohol to be subjected to the collecting step is a gas-liquid mixture containing isopropyl alcohol and a gas, the gas mainly containing a gas such as hydrogen and a liquid mixture containing isopropyl alcohol are separated from each other by a known gas-liquid separation method, followed by collecting isopropyl alcohol. Here, the gas refers to a substance that exists in a gaseous state under pressurized and cooled conditions in the gas-liquid separation operation.

In the collecting step, the pressure in the gas-liquid separation operation is preferably 0.1 MPa to 2 MPa, more preferably 0.2 MPa to 1 MPa.

In the collecting step, the temperature in the gas-liquid separation operation is preferably 0° C. to 50° C., more preferably 5° C. to 40° C.

Distillation of the liquid mixture containing isopropyl alcohol obtained by the gas-liquid separation operation in the collecting step can provide purified isopropyl alcohol. The liquid mixture containing isopropyl alcohol can be distilled by a known distillation method. Examples of the known distillation method include thin-film distillation and rectification. The distillation operation may be continuous or batchwise.

In the collecting step, the distillation operation may be azeotropic distillation. Isopropyl alcohol and water form an azeotrope. Thus, when the liquid mixture containing isopropyl alcohol contains water, azeotropic distillation of the liquid mixture can provide high-purity isopropyl alcohol.

### (Gas Separating Step)

The method for producing isopropyl alcohol of the present disclosure may include a gas separating step. An example of the gas separating step is purifying hydrogen in the gas phase component obtained in the step (2) of purifying acetone (gas-liquid separating step). Hereinafter, the purifying hydrogen in the gas phase component obtained in the step (2) is also referred to as a step (4).

A hydrogen-rich composition obtained in the step (4) may be directly collected, or a portion of or the entire composition may be used for acetone reduction in the step (3).

The hydrogen/carbon dioxide molar ratio of the hydrogen-rich composition obtained in the step (4) is preferably 90:10 or more, more preferably 95:5 or more, still more preferably 98:2 or more.

Introducing such a hydrogen-rich composition with a low carbon dioxide content into the step (3) tends to improve catalytic activity.

Examples of the method of gas separation (method for purifying hydrogen) in the step (4) include known methods such as a physical absorption method, a chemical absorption method, a membrane separation method, a cryogenic separation method, and a compression liquefaction method.

The physical absorption method is a method of separating and collecting carbon dioxide from a mixed gas by physical action such as adsorption or dissolution without performing a chemical reaction. The method is particularly preferably the pressure swing adsorption (PSA) process.

In the chemical absorption method, carbon dioxide is reacted mainly with a basic substance such as an amine or alkali to be converted to a hydrogen carbonate for absorption. Carbon dioxide can be separated and collected from the absorption liquid by heating or decompression of the absorption liquid.

The membrane separation method is preferably a method using a separation membrane that selectively permeates hydrogen or carbon dioxide. Non-limiting examples of the membrane used in the method include polymer membranes, dendrimer membranes, amine group-containing membranes, and inorganic membranes including zeolite membranes. The separation membrane may contain a metal atom. The metal atom is not limited, and may be Pd, for example.

The step (4) preferably includes at least one step selected from membrane separation, absorption into a basic substance or organic solvent, and adsorption into an adsorbent such as PSA or activated carbon.

### <Embodiment of Method for Producing Isopropyl Alcohol>

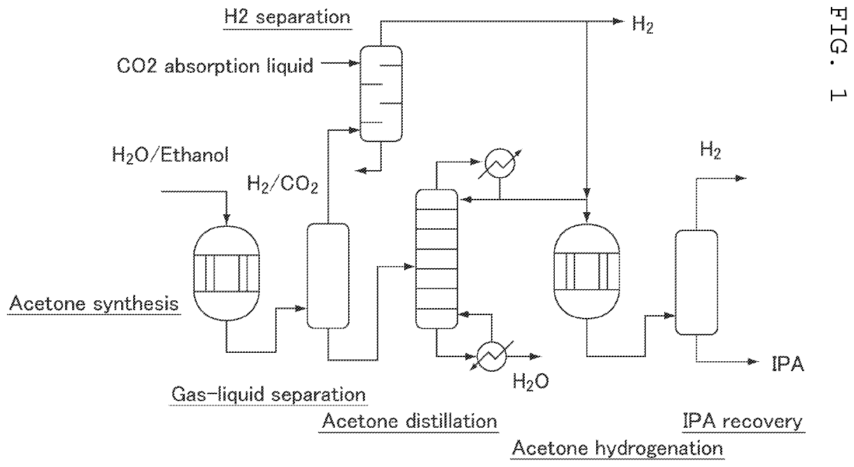

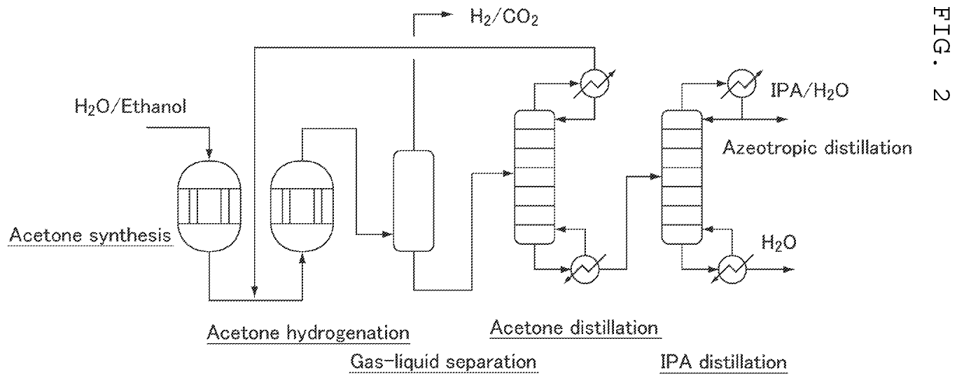

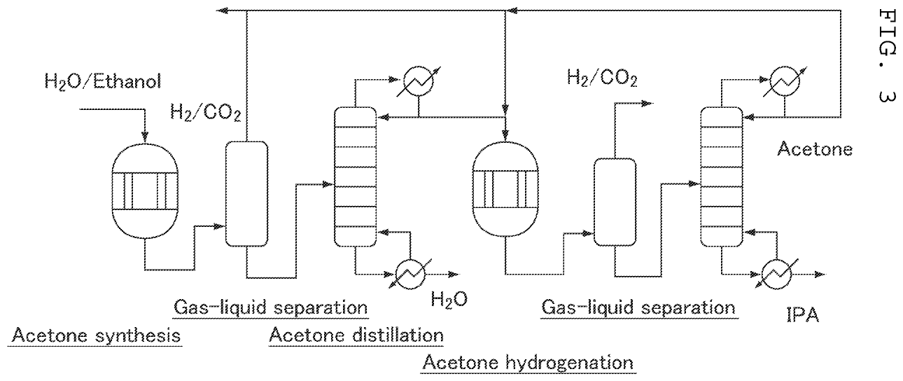

The following describes the exemplary preferred embodiments of the production process of isopropyl alcohol in the present disclosure. The processes shown in FIGS. 1 and 3 are each an exemplary process in which the steps (1), (2), and (3) are performed in this order, followed by an isopropyl alcohol collecting step. The process shown in FIG. 2 is an exemplary process in which the steps (1), (3), and (2) are performed in this order, followed by an isopropyl alcohol collecting step. The process shown in FIG. 1 further includes a gas separating step.

For obtaining high-purity isopropyl alcohol, the process shown in FIG. 2 may include an azeotropic distillation step of isopropyl alcohol in the isopropyl alcohol collecting step.

Among the production processes shown in FIGS. 1 to 3, the processes shown in FIGS. 1 and 3 are more preferred from the viewpoint of simple production. From the viewpoint of catalyst activity, the process shown in FIG. 1 is more preferred in which the amount of carbon dioxide to be introduced into the step (3) is small.

### (Catalyst Regenerating Step)

The method for producing isopropyl alcohol of the present disclosure may include a catalyst regenerating step when the activity of the catalyst has changed.

The regenerating may be performed by any method such as a method of bringing the catalyst into contact with an oxidizing gas such as oxygen at a high temperature. For example, in the case where a feed gas is supplied to a fixed-bed reactor for production of isopropyl alcohol, the catalyst may be regenerated using an oxidizing gas instead of the feed gas, or the catalyst may be regenerated after being removed from the reactor.

### (Isopropyl Alcohol Production Apparatus in the Present Disclosure)

A production apparatus used for the production of isopropyl alcohol in the present disclosure may be either batchwise or continuous. From the viewpoint of productivity, the production apparatus is preferably continuous.

A continuous apparatus for performing the step (1) may include a known reactor such as a fixed-bed reactor, a fluidized-bed reactor, or a moving-bed reactor. Preferred among these is a fixed-bed reactor in terms of simpler equipment and easier operation.

Among the apparatuses for performing the step (2), a gas-liquid separator is not limited, and may be a common gas-liquid separator having a pressurization/cooling mechanism. A distillation apparatus is not limited, and is preferably a distillation apparatus including a distillation column with a theoretical number of plates of 4 to 40.

A continuous apparatus for performing the step (3) may include a known reactor such as a fixed-bed reactor, a fluidized-bed reactor, or a moving-bed reactor. Preferred among these is a fixed-bed reactor in terms of simpler equipment and easier operation.

### (Isopropyl Alcohol Obtained by the Production Method of the Present Disclosure)

The production method of the present disclosure preferably provides isopropyl alcohol containing ethanol, water, and acetone as impurities, each of which has a concentration of 10,000 ppm or less. Such isopropyl alcohol containing impurities in low concentrations can be suitably used in various industrial applications. The concentrations of ethanol, water, and acetone as impurities are each more preferably 10,000 ppm or less, still more preferably 5,000 ppm or less.

From the viewpoint of reducing the amount of impurities, the processes shown in FIGS. 1 and 3 are more preferred. From the viewpoint of the number of steps, the process shown in FIG. 1 is more preferred.

### (Uses of Isopropyl Alcohol Obtained by the Production Method of the Present Disclosure)

Uses of isopropyl alcohol obtained by the production method of the present disclosure are not limited, and the isopropyl alcohol can be suitably used as a raw material for producing propylene. Propylene can be produced by dehydrating the isopropyl alcohol in the present disclosure by a known method, for example.

### EXAMPLES

The present invention is described in more detail below with reference to examples, but the present invention is not limited to these examples. It should be noted that the terms “part(s)” and “%” refer to “part(s) by weight” and “% by mass”, respectively, unless otherwise stated.

### <Synthesis of Catalyst>

### (Preparation of Catalyst A1/Acetone Synthesis Catalyst)

In 400 mL of pure water was dissolved 12.3 g of zinc nitrate hexahydrate (FUJIFILM Wako Pure Chemical Corporation, purity 99.0% or higher), 4.7 g of aluminum nitrate hexahydrate (FUJIFILM Wako Pure Chemical Corporation, purity 97.0% or higher), and 33.4 g of iron nitrate nonahydrate (Nacalai Tesque, Inc., purity 98.0% or higher) to prepare an aqueous solution mixture containing zinc nitrate, iron nitrate, and aluminum nitrate. While the aqueous solution mixture was stirred with a magnetic stirrer, an aqueous ammonia solution (FUJIFILM Wako Pure Chemical Corporation, purity 28.0%) was added dropwise thereto at room temperature to adjust the pH to 8. The obtained precipitate was collected by filtration, dried at 120° C. for 10 hours, and then fired at 450° C. for 2 hours to obtain a catalyst A1.

### (Preparation of Catalyst B1/Acetone Hydrogenation Catalyst)

To 4.0 g of pure water was added 0.4 mL of a dinitrodiammine platinum nitrate solution (Tanaka Kikinzoku Kogyo K.K., platinum content 100 g/L) to prepare a platinum-containing aqueous solution. The platinum-containing aqueous solution was added to 4.0 g of cerium oxide powder (Rhodia, 3CO) weighed in a magnetic dish, and they were mixed uniformly with a glass rod. Subsequently, the mixture was dried at 120° C. for 10 hours and fired at 400° C. for 1 hour to obtain a catalyst B1.

### Example 1

### (Step (1): Acetone Synthesis Reaction)

An acetone synthesis reaction was performed using a SUS316 tubular reactor (outer diameter 10 mm, inner diameter 8 mm). A catalyst A1 powder, which was uniformly ground in an agate mortar, was packed into a vinyl chloride disk, and compressed with a compression molding machine at 30 MPaG into a disk shape. The disk-shaped workpiece was crushed and particles having sizes of 0.71 to 1.18 mm were separated, and a 2.0 g-portion of the particulate catalyst was packed into the reaction tube made of stainless steel. The reaction tube filled with the catalyst A1 was placed in a circular electric furnace, and nitrogen was supplied thereto at 50.0 mL/min (in terms of 0° C. and 1 atm). The temperature was increased by heating with the electric furnace to 400° C. and kept for 30 minutes. Thereafter, the nitrogen supply was stopped, a 56.1% by weight ethanol aqueous solution was supplied at 0.08 g/min by a feeder. The reaction was performed at atmospheric pressure.

When the ethanol aqueous solution was supplied into the reaction system by the feeder, the solution was supplied into an ethanol-aqueous-solution vaporization section provided on the inlet side of the reaction tube. The ethanol-aqueous-solution vaporization section was heated to 100° C. by external heating. The ethanol aqueous solution supplied in the form of liquid to the ethanol-aqueous-solution vaporization section by the feeder was immediately vaporized and introduced into the SUS316 tubular reactor.

The gas discharged from the outlet of the reaction tube was analyzed and found to have an ethanol conversion rate of 100% and an acetone selectivity of 69%.

Here, the ethanol conversion rate and acetone yield were calculated using the following equations (1) and (2).

$$Ethanol conversion rate = 100 - 1 \; 00 \times ( Ethanol flow rate at outlet of reactor / Ethanol flow rate at inlet of reactor ) \qquad ( 1 )$$

$$Acetone yield = 100 \times Acetone flow rate at outlet of reactor \times 3 / ( Ethanol flow rate at inlet of reactor \times 2 ) \qquad ( 2 )$$

The acetone synthesis reaction of ethanol and water is represented by the following reaction formula (3).

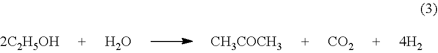

The acetone yield represented by the formula (2) was evaluated based on the amount of carbon in the produced acetone relative to the total amount of carbon in the ethanol supplied from the inlet of the reactor. Thus, the upper limit of the acetone yield is 75%.

### (Step (2): Acetone Purification)

The gas discharged from the outlet of the reactor obtained in the step (1) was introduced into a glass absorption bottle cooled to ice temperature. The gas was bubbled through ice-temperature pure water in the glass absorption bottle. Thereby, condensed components containing acetone and water in the gas discharged from the outlet of the reactor were collected. The collected liquid was analyzed by gas chromatography to found that most of the collected components other than water were acetone, with only a trace amount of structurally unknown components being present in the collected liquid.

The separation and purification of acetone from the aqueous acetone solution can be performed by typical distillation operation. Thus, by distilling the obtained aqueous acetone solution under appropriate conditions, high-purity acetone can be obtained from the top of the distillation column of the distillation apparatus.

A component not condensed and collected in the absorption bottle was discharged as a gas component from the absorption bottle. The gas component contained hydrogen and carbon dioxide as main components, with small amounts of hydrocarbons such as methane, ethylene, and ethane, which were by-products of the reaction, being detected as other components. This demonstrated that the amount of acetone in the gas component was very small, and that almost total amount of the acetone produced in the reaction process was collected in the previous absorption operation.

The gas component was passed through a 10% by weight aqueous sodium hydroxide solution in the absorption bottle at room temperature for absorption of carbon dioxide in the gas. Subsequently, the gas component was passed through an activated carbon-filled column so that hydrocarbons such as methane, ethylene, and ethane were removed by adsorption. Thereby, purified hydrogen gas was obtained.

### (Step (3): Acetone Hydrogenation Reaction)

An acetone hydrogenation reaction was performed under the following conditions. Reagent acetone (Nacalai Tesque, Inc., purity 99.5% or higher) was used instead of purified acetone obtained by distilling the acetone aqueous solution obtained by absorption of the gas discharged from the outlet of the acetone synthesis reactor. Also, as for hydrogen, instead of using high-purity hydrogen obtained by treating the gas discharged from the outlet of the acetone synthesis reactor, hydrogen was supplied from a hydrogen cylinder (NIPPON STEEL Chemical & Material CO., LTD., purity 99.999% or higher).

The acetone hydrogenation reaction was performed using a SUS316 tubular reactor (outer diameter 10 mm, inner diameter 8 mm). A catalyst B1 powder, which was obtained by uniformly grinding the catalyst B1 in an agate mortar, was packed into a vinyl chloride disk, and compressed with a compression molding machine at 30 MPaG into a disk shape. The disk-shaped workpiece was crushed. Particles having sizes of 0.71 to 1.18 mm were separated, and a 1.4 g-portion of the particulate catalyst was packed into the reaction tube made of stainless steel.

The reaction tube filled with the catalyst B1 was placed in a circular electric furnace, and hydrogen and nitrogen were supplied at 10.0 mL/min (in terms of 0° C. and 1 atm) and at 40.0 mL/min (in terms of 0° C. and 1 atm), respectively. The temperature was increased by heating with the electric furnace to 300° C. and kept for 30 minutes. Thus, the catalyst B1 was reduced. Thereafter, the temperature was lowered to 40° C., the nitrogen supply was stopped, hydrogen supply was started at 60 mL/min (in terms of 0° C. and 1 atm), and acetone was supplied at 0.08 g/min by a feeder. The reaction was performed at atmospheric pressure. The molar ratio of hydrogen/acetone in the inlet gas was 2.

When the acetone was supplied into the reaction system by the feeder, the acetone was supplied into an acetone-vaporization section provided on the inlet side of the reaction tube. The acetone-vaporization section was heated to 40° C. by external heating, and hydrogen was supplied to the vaporization section from the upstream side. The acetone supplied in the form of liquid to the acetone-vaporization section by the feeder was immediately vaporized. The gas was entrained into and mixed with hydrogen and introduced into the SUS316 tubular reactor.

The gas discharged from the outlet of the reaction tube was analyzed and found to have an acetone conversion rate of 98.9% and an isopropyl alcohol selectivity of 99.9%.

Here, the acetone conversion rate and isopropyl alcohol selectivity were calculated using the following equations (4) and (5).

$$Acetone conversion rate = 100 - 100 \times ( Acetone flow rate at outlet of reactor / Acetone flow rate at inlet of reactor ) \qquad ( 4 )$$

$$Isopropyl alcohol selectivity = 100 \times Isopropyl alcohol flow rate at outlet of reactor / ( Acetone flow rate at inlet of reactor - Acetone flow rate at outlet of reactor ) \qquad ( 5 )$$

The gas component discharged from the outlet of the acetone hydrogenation reactor contains hydrogen, isopropyl alcohol, and a trace amount of acetone. Thus, isopropyl alcohol and hydrogen can be easily separated by typical gas-liquid separation under pressurizing and cooling.

These results demonstrate that isopropyl alcohol can be efficiently produced from ethanol and water by the process shown in FIG. 1.

### Example 2

### (Step (3): Acetone Hydrogenation Reaction)

An acetone hydrogenation reaction was performed under the conditions corresponding to the conditions of the case where the hydrogen used (in the acetone hydrogenation reaction) in Example 1 was changed to the gas discharged from the absorption bottle after acetone collection and mainly containing hydrogen and carbon dioxide.

The gas discharged from the absorption bottle after acetone collection had a composition mainly containing hydrogen and carbon dioxide, and the molar ratio of hydrogen/carbon dioxide is approximately 4/1. A simulated gas having the same molar ratio as the above molar ratio of the gas discharged from the absorption bottle was prepared using a hydrogen cylinder (NIPPON STEEL Chemical & Material CO., LTD., purity 99.999% or higher) and a liquefied carbon dioxide cylinder (Sumitomo Seika Chemicals Company, Limited., purity 99.9% or higher).

An acetone hydrogenation reaction was performed as in Example 1, except that supplying of hydrogen at 48 mL/min (in terms of 0° C. and 1 atm) and carbon dioxide at 12 mL/min (in terms of 0° C. and 1 atm), totaling 60 mL/min, was performed instead of supplying of hydrogen at 60 mL/min (in terms of 0° C. and 1 atm).

The gas discharged from the outlet of the reaction tube was analyzed and found to have an acetone conversion rate of 9.4% and an isopropyl alcohol selectivity of 99.3%.

Since in Example 2, supplying of hydrogen at 48 mL/min (in terms of 0° C. and 1 atm) and carbon dioxide at 12 mL/min (in terms of 0° C. and 1 atm) was performed instead of supplying of hydrogen at 60 mL/min (in terms of 0° C. and 1 atm) in Example 1, the molar ratio of hydrogen/acetone in the inlet gas was reduced from 2 in Example 1 to 1.6 in Example 2. This decrease in the hydrogen/acetone molar ratio is presumably a factor in the significant decrease in the acetone conversion rate. However, in another acetone hydrogenation reaction similarly performed in which supplying of hydrogen at 48 mL/min (in terms of 0° C. and 1 atm) and nitrogen at 12 mL/min (in terms of 0° C. and 1 atm), totaling 60 mL/min (in terms of 0° C. and 1 atm), was performed, the same reaction results as those in Example 1 were obtained. Thus, the decrease in the acetone conversion rate in Example 2 was not caused by the decrease in the hydrogen/acetone molar ratio, but was caused by the significantly inhibited acetone hydrogenation reaction by coexistence of carbon dioxide.

Examples 1 and 2 demonstrated that, in the acetone hydrogenation reaction using a 1% by mass Pt/CeO₂ catalyst, the coexistence of carbon dioxide significantly inhibited the acetone hydrogenation reaction. In response to this, in order to investigate acetone hydrogenation catalysts functioning effectively even in the coexistence of carbon dioxide, catalysts B2 to B10 were prepared. The acetone hydrogenation activities thereof in the coexistence of 15% by volume of carbon dioxide were compared to each other for evaluation (Experimental Examples 1 to 9).

### (Preparation of Catalyst B2/Acetone Hydrogenation Catalyst)

Pure water was added to 0.49 g of a dinitrodiammine platinum nitrate solution (Tanaka Kikinzoku Kogyo K.K., Pt content 8.19% by mass) in a beaker to prepare a platinum-containing aqueous solution. The platinum-containing aqueous solution was added to 4 g of ZrO₂ powder (Daiichi Kigenso Kagaku Kogyo Co., Ltd., EP-L, specific surface area 102 m²/g) in a magnetic dish, and they were heated while being stirred with a glass rod to vaporize water. The obtained powder was dried at 120° C. for 10 hours and fired at 400° C. for 1 hour to prepare a catalyst B2. The resulting catalyst B2 had a composition of 1% by mass Pt/ZrO₂ (i.e., 1% by mass Pt relative to 99% by mass ZrO₂).

### (Preparation of Catalyst B3/Acetone Hydrogenation Catalyst)

A catalyst B3 was prepared as in Preparation of catalyst B2, except that 0.49 g of the dinitrodiammine platinum nitric acid solution was changed to 1.03 g of a ruthenium nitrate solution (Tanaka Kikinzoku Kogyo K.K., Ru content 3.92% by mass). The obtained catalyst B3 had a composition of 5% by mass Ru/ZrO₂.

### (Preparation of Catalyst B4/Acetone Hydrogenation Catalyst)

A catalyst B4 was prepared as in Preparation of catalyst B2, except that 0.49 g of the dinitrodiammine platinum nitric acid solution was changed to 11.3 g of a ruthenium nitrate solution (Tanaka Kikinzoku Kogyo K.K., Ru content 3.92% by mass). The obtained catalyst B4 had a composition of 10% by mass Ru/ZrO₂.

### (Preparation of Catalyst B5/Acetone Hydrogenation Catalyst)

A catalyst B5 was prepared as in Preparation of catalyst B2, except that 0.49 g of the dinitrodiammine platinum nitric acid solution was changed to 1.24 g of nickel nitrate hexahydrate (Nacalai Tesque, Inc., special grade). The obtained catalyst B5 had a composition of 5.9% by mass Ni/ZrO₂.

### (Preparation of Catalyst B6/Acetone Hydrogenation Catalyst)

A catalyst B6 was prepared as in Preparation of catalyst B2, except that 0.49 g of the dinitrodiammine platinum nitrate solution was changed to a combination of 0.36 g of nickel nitrate hexahydrate (Nacalai Tesque, Inc., special grade) and 0.49 g of a dinitrodiammine platinum nitrate solution (Tanaka Kikinzoku Kogyo K.K., Pt content 8.19% by mass). The obtained catalyst B6 had a composition of 1.8% by mass Ni/1% by mass Pt/ZrO₂.

### (Preparation of Catalyst B7/Acetone Hydrogenation Catalyst)

Pure water was added to 1.10 g of nickel nitrate hexahydrate (Nacalai Tesque, Inc. Co., Ltd., special grade) and 5.67 g of a ruthenium nitrate solution (Tanaka Kikinzoku Kogyo K.K., Ru content 3.92% by mass) to prepare an aqueous solution mixture containing nickel and ruthenium. The aqueous solution mixture was added to 4 g of ZrO₂ powder (Daiichi Kigenso Kagaku Kogyo Co., Ltd., EP-L, specific surface area 102 m²/g) in a magnetic dish, and they were heated while being stirred with a glass rod to vaporize water. The obtained powder was dried at 120° C. for 10 hours and then fired at 400° C. for 1 hour to prepare a catalyst B7. The obtained catalyst B7 had a composition of 5% by mass Ni/5% by mass Ru/ZrO₂.

### (Preparation of Catalyst B8/Acetone Hydrogenation Catalyst)

A catalyst B8 was prepared as in Preparation of catalyst B7, except that the ZrO₂ powder (Daiichi Kigenso Kagaku Kogyo Co., Ltd., EP-L, specific surface area 102 m²/g) was changed to ZrO₂ powder (Daiichi Kigenso Kagaku Kogyo Co., Ltd. RC-100, specific surface area 118 m²/g). The obtained catalyst B8 had a composition of 5% by mass Ni/5% by mass Ru/ZrO₂.

### (Preparation of Catalyst B9/Acetone Hydrogenation Catalyst)

A catalyst B9 was prepared as in Preparation of catalyst B7, except that the ZrO₂ powder (Daiichi Kigenso Kagaku Kogyo Co., Ltd., EP-L, specific surface area 102 m²/g) was changed to CeO₂ powder (Rhodia, 3CO, specific surface area 171 m²/g). The obtained catalyst B9 had a composition of 5% by mass Ni/5% by mass Ru/CeO₂.

### (Preparation of Catalyst B10/Acetone Hydrogenation Catalyst)

A catalyst B10 was prepared as in Preparation of catalyst B7, except that the ZrO₂ powder (Daiichi Kigenso Kagaku Kogyo Co., Ltd., EP-L, specific surface area 102 m²/g) was changed to SiO₂ powder (Fuji Silysia Chemical Ltd., Cariact Q-6, specific surface area 113 m²/g). The obtained catalyst B10 had a composition of 5% by mass Ni/5% by mass Ru/SiO₂.

### Experimental Example 1

An acetone hydrogenation reaction was performed using a SUS316 tubular reactor (outer diameter 10 mm, inner diameter 8 mm). A catalyst B2 powder, which was obtained by uniformly grinding the catalyst B2 in an agate mortar, was packed into a vinyl chloride disk, and compressed with a compression molding machine at 30 MPaG into a disk shape. The disk-shaped workpiece was crushed. Particles having sizes of 0.71 to 1.18 mm were separated, and a 0.35 g-portion of the particulate catalyst was packed into the reaction tube made of stainless steel. The catalyst was pretreated at 300° C. for one hour while nitrogen (N₂) and hydrogen (H₂) were flowed at 20 cm³/min (flow rate at standard conditions of 0° C. and 1 atm) and at 15 cm³/min (flow rate at standard conditions of 0° C. and 1 atm), respectively. Subsequently, after setting the reaction temperature, carbon dioxide (CO₂) was added at 7.5 cm³/min (flow rate at standard conditions of 0° C. and 1 atm) to prepare a gas mixture flow consisting of nitrogen, hydrogen, and carbon dioxide. This gas mixture flow was introduced into a bubbler containing pure water at 25° C., and water vapor equivalent to saturated water vapor was entrained in the flow. Additional acetone (Nacalai Tesque, Inc., special grade) was introduced into the gas mixture flow, coming out of the bubbler, consisting of nitrogen, hydrogen, carbon dioxide, and water using a microsyringe feeder at 19.4 mg/min. Thereby, an isopropyl alcohol production reaction involving acetone hydrogenation in the coexistence of carbon dioxide was started.

The gas discharged from the outlet of the reactor was introduced into a trap placed in an ice water bath, and unreacted raw materials and products were collected in the trap. The liquid component collected in the trap was quantitatively analyzed by GC-FID (Agilent, 7890B/capillary column HP-plot Q). The gas product not collected in the trap was directly introduced into GC-FID and analyzed. From these analysis results, the acetone conversion rate and isopropyl alcohol selectivity were calculated using the equations 3 and 4. Table 1 shows the reaction results obtained at an electric furnace temperature of 100° C.

### Experimental Example 2

An isopropyl alcohol production reaction involving acetone hydrogenation in the coexistence of carbon dioxide was started as in Experimental Example 1, except that the catalyst B2 was changed to the catalyst B3. Table 1 shows the reaction results obtained at an electric furnace temperature of 100° C.

### Experimental Example 3

An isopropyl alcohol production reaction involving acetone hydrogenation in the coexistence of carbon dioxide was started as in Experimental Example 1, except that the catalyst B2 was changed to the catalyst B4. Table 1 shows the reaction results obtained at an electric furnace temperature of 100° C.

### Experimental Example 4

An isopropyl alcohol production reaction involving acetone hydrogenation in the coexistence of carbon dioxide was started as in Experimental Example 1, except that the catalyst B2 was changed to the catalyst B5. Table 1 shows the reaction results obtained at an electric furnace temperature of 100° C.

### Experimental Example 5

An isopropyl alcohol production reaction involving acetone hydrogenation in the coexistence of carbon dioxide was started as in Experimental Example 1, except that the catalyst B2 was changed to the catalyst B6. Table 1 shows the reaction results obtained at an electric furnace temperature of 100° C.

### Experimental Example 6

An isopropyl alcohol production reaction involving acetone hydrogenation in the coexistence of carbon dioxide was started as in Experimental Example 1, except that the catalyst B2 was changed to the catalyst B7. Table 1 shows the reaction results obtained at an electric furnace temperature of 100° C.

### Experimental Example 7

An isopropyl alcohol production reaction involving acetone hydrogenation in the coexistence of carbon dioxide was started as in Experimental Example 1, except that the catalyst B2 was changed to the catalyst B8. Table 1 shows the reaction results obtained at an electric furnace temperature of 100° C.

### Experimental Example 8

An isopropyl alcohol production reaction involving acetone hydrogenation in the coexistence of carbon dioxide was started as in Experimental Example 1, except that the catalyst B2 was changed to the catalyst B9. Table 1 shows the reaction results obtained at an electric furnace temperature of 100° C.

### Experimental Example 9

An isopropyl alcohol production reaction involving acetone hydrogenation in the coexistence of carbon dioxide was started as in Experimental Example 1, except that the catalyst B2 was changed to the catalyst B10. Table 1 shows the reaction results obtained at an electric furnace temperature of 100° C.

TABLE 1

| | Catalyst | Conversion rate (%) | IPA selectivity (%) |
|---|---|---|---|
| Experimental | Catalyst B2 | 24.2 | 91.7 |
| Example 1 | | | |
| Experimental | Catalyst B3 | 55.1 | 98.5 |
| Example 2 | | | |
| Experimental | Catalyst B4 | 69.0 | 98.8 |
| Example 3 | | | |
| Experimental | Catalyst B5 | 14.7 | 77.2 |
| Example 4 | | | |
| Experimental | Catalyst B6 | 40.6 | 99.5 |
| Example 5 | | | |
| Experimental | Catalyst B7 | 66.8 | 99.9 |
| Example 6 | | | |
| Experimental | Catalyst B8 | 84.9 | 99.9 |
| Example 7 | | | |
| Experimental | Catalyst B9 | 58.9 | 99.9 |
| Example 8 | | | |
| Experimental | Catalyst B10 | 80.5 | 99.9 |
| Example 9 | | | |

Even when the CeO₂ support of the 1% by mass Pt/CeO₂ catalyst tested in Examples 1 and 2 was changed to a ZrO₂ support (the catalyst B2), the activity inhibition effect of carbon dioxide did not change significantly, and the conversion rate was 24.2% (Experimental Example 1). When the catalyst B3 containing 5% by mass of ruthenium as a metal component (5% by mass Ru/ZrO₂) was used, the acetone conversion rate was 55.1%. The catalyst B4 in which the amount of ruthenium supported was increased to 10% by mass (10% by mass Ru/ZrO₂) exhibited an increased acetone conversion rate of 69.0%.

The catalyst B5 containing only 5.9% by mass of nickel (5.9% by mass Ni/ZrO₂) exhibited an acetone conversion rate as low as 14.7%.

The catalyst B6 containing 1.8% by mass of nickel and 1% by mass of platinum exhibited an acetone conversion rate of 40.6%, which was insufficient.

The catalyst B7 in which 5% by mass of ruthenium and 5% by mass of nickel were supported on ZrO₂ (EP-L) provides better acetone hydrogenation activity than the catalyst B3 in which only 5% by mass of ruthenium was supported and the catalyst B5 in which only 5.9% by mass of nickel was supported. Also, the acetone conversion rate was 66.8%. Also, high acetone conversion rates were achieved also in the cases of the catalysts B8, B9, and B10 in which 5% by mass (fixed amount) of ruthenium and 5% by mass (fixed amount) of nickel were supported on the supports ZrO₂ (RC-100), CeO₂, and SiO₂, respectively.

Next, catalysts A2 to A4 were prepared as acetone synthesis catalysts for synthesizing acetone from ethanol, and the acetone synthesis reaction was continuously performed at 375° C. to test the stability of the catalysts (Experimental Examples 10 to 12).

### Preparation Example of Catalyst A2/Acetone Synthesis Catalyst

In 400 mL of pure water was dissolved 12.3 g of zinc nitrate hexahydrate (FUJIFILM Wako Pure Chemical Corporation, purity 99.0% or higher), 22.3 g of zirconium oxynitrate hydrate (Aldrich, technical grade), and 33.4 g of iron nitrate nonahydrate (Nacalai Tesque, Inc., purity 98.0% or higher) to prepare an aqueous solution mixture containing zinc nitrate, iron nitrate, and zirconium oxynitrate. While the aqueous solution mixture was stirred with a magnetic stirrer, an aqueous ammonia solution (FUJIFILM Wako Pure Chemical Corporation, purity 28.0%) was added dropwise thereto at room temperature to adjust the pH to 8. The obtained precipitate was collected by filtration, dried at 120° C. for 10 hours, and then fired at 450° C. for 2 hours to obtain a catalyst A2.

The catalyst A2 had a composition of Fe₂O₃/ZnO/ZrO₂=32.6/16.6/50.8 (% by mass).

### Preparation Example of Catalyst A3/Acetone Synthesis Catalyst

A catalyst A3 was obtained as in Preparation example of catalyst A2, except that the amount of zirconium oxynitrate hydrate (Aldrich, technical grade) was changed from 22.3 g to 3.32 g. The catalyst A3 had a composition of Fe₂O₃/ZnO/ZrO₂=57.4/29.3/13.3 (% by mass).

### Preparation Example of Catalyst A4/Acetone Synthesis Catalyst

A catalyst A4 was obtained as in Preparation example of catalyst A2, except that the amount of zirconium oxynitrate hydrate (Aldrich, technical grade) was changed from 22.3 g to 11.1 g. The catalyst A4 had a composition of Fe₂O₃/ZnO/ZrO₂=43.8/22.3/33.9 (% by mass).

### Experiment Example 10

An acetone synthesis reaction was performed using a SUS316 tubular reactor (outer diameter 10 mm, inner diameter 8 mm). A catalyst A2 powder, which was obtained by uniformly grinding the catalyst A2 in an agate mortar, was packed into a vinyl chloride disk, and compressed with a compression molding machine at 30 MPaG into a disk shape. The disk-shaped workpiece was crushed. Particles having sizes of 0.71 to 1.18 mm were separated, and a 1.4 g-portion of the particulate catalyst was packed into the reaction tube made of stainless steel. The reaction tube filled with the catalyst A2 was placed in a circular electric furnace, and nitrogen was supplied thereto at 11.0 mL/min (in terms of 0° C. and 1 atm). The temperature was increased by heating with the electric furnace to 375° C. and kept for 30 minutes. Thereafter, a 56.1% by weight ethanol aqueous solution was additionally supplied at 0.056 g/min by a feeder. The reaction was performed at atmospheric pressure.

When the ethanol aqueous solution was supplied into the reaction system by the feeder, the solution was supplied into an ethanol-aqueous-solution vaporization section provided on the inlet side of the reaction tube. The ethanol-aqueous-solution vaporization section was heated to 100° C. by external heating. The ethanol aqueous solution supplied in the form of liquid to the ethanol-aqueous-solution vaporization section by the feeder was immediately vaporized and introduced into the SUS316 tubular reactor together with nitrogen.

In the continuous tests, since the reaction temperature was set at 375° C., the production of acetaldehyde, which is an intermediate product, in addition to acetone was also confirmed. In the continuous tests, stability evaluation was performed by tracking changes over time in ethanol conversion, acetone selectivity, and acetaldehyde selectivity. The acetone selectivity and acetaldehyde selectivity were calculated as follows. The ethanol conversion rate was calculated using the formula (1) described in Example 1. Table 2 summarizes the results of a 528-hour reaction.

$$Acetone selectivity = 100 \times Flow rate of acetone at outlet of reactor \times 3 / ( Flow rate of ethanol at inlet of reactor \times 2 \times Ethanol conversion rate \times 0.01 )$$

$$Acetaldehyde selectivity = 100 \times Flow rate of acetaldehyde at outlet of reactor / ( Flow rate of ethanol at inlet of reactor \times Ethanol conversion rate \times 0.01 )$$

TABLE 2

| Reaction | Ethanol | Acetone | Acetaldehyde |
|---|---|---|---|
| time (h) | conversion (%) | selectivity (%) | selectivity (%) |
| 1 | 49.0 | 41.8 | 18.3 |
| 20 | 38.6 | 32.3 | 27.7 |
| 144 | 37.5 | 32.8 | 30.3 |
| 168 | 39.3 | 31.9 | 28.7 |
| 192 | 37.5 | 34.1 | 29.8 |
| 216 | 40.3 | 35.2 | 27.7 |
| 312 | 38.1 | 35.1 | 27.8 |
| 384 | 38.9 | 36.2 | 27.2 |
| 504 | 39.5 | 36.3 | 26.0 |
| 528 | 38.3 | 34.6 | 26.6 |

### Experimental Example 11

An acetone synthesis reaction was performed as in Experimental Example 10, except that the catalyst A2 was changed to the catalyst A3. Table 3 summarizes the results of a 284-hour reaction.

TABLE 3

| Reaction | Ethanol | Acetone | Acetaldehyde |
|---|---|---|---|
| time (h) | conversion (%) | selectivity (%) | selectivity (%) |
| 1.5 | 54.0 | 53.6 | 7.8 |
| 43.5 | 46.1 | 47.5 | 13.7 |
| 115.5 | 40.2 | 48.4 | 15.2 |
| 188 | 36.6 | 46.4 | 16.9 |
| 284 | 33.1 | 43.3 | 19.4 |

### Experimental Example 12

An acetone synthesis reaction was performed as in Experimental Example 10, except that the catalyst A2 was changed to the catalyst A4. Table 4 summarizes the results of a 312-hour reaction.

TABLE 4

| Reaction | Ethanol | Acetone | Acetaldehyde |
|---|---|---|---|
| time (h) | conversion (%) | selectivity (%) | selectivity (%) |
| 1 | 51.6 | 47.1 | 14.5 |
| 24 | 43.4 | 38.7 | 22.4 |
| 72 | 45.4 | 42.1 | 21.0 |
| 144 | 47.9 | 44.8 | 17.1 |
| 240 | 49.5 | 47.7 | 15.0 |
| 312 | 49.2 | 47.2 | 14.7 |

Since the catalyst A3 had a low ZrO₂ content, the catalytic activity thereof decreased in a short period of time. In the cases of the catalysts A2 and A4, each of which having a higher ZrO₂ content, a decrease in catalytic activity over time was prevented. In the case of the catalyst A4, the conversion rate decreased from 51.6% to 43.4% at the beginning of the reaction, followed by recovery of the activity of the catalyst A4. This behavior was also reproduced in another test performed under the same conditions. These results demonstrated that acetone can be stably synthesized from ethanol by using a Fe₂O₃/ZnO/ZrO₂ catalyst containing a predetermined amount of ZrO₂.

Here, in the tests, in order to track the behavior of change in catalyst performance in the continuous tests, the reaction was targeted at a medium conversion rate. Thus, a large amount of acetaldehyde as an intermediate product was detected. Alternatively, the operation can be performed to achieve a high ethanol conversion and a high acetone selectivity by increasing the amount of catalyst, increasing the reaction temperature, or changing other conditions.

1. A method for producing isopropyl alcohol, the method comprising:
a step (1) of reacting ethanol and water in the presence of a catalyst to obtain acetone;
a step (2) of purifying the acetone; and
a step (3) of reducing the acetone to obtain isopropyl alcohol.

2. The method for producing isopropyl alcohol according to claim 1,
wherein a reducing agent used in the step (3) includes hydrogen obtained in the step (1).

3. The method for producing isopropyl alcohol according to claim 1, wherein a reducing agent used in the step (3) includes hydrogen obtained in the step (2).

4. The method for producing isopropyl alcohol according to claim 1,
wherein a percentage of an amount of carbon dioxide in all components introduced in the step (3) is less than 10 mol %.

5. The method for producing isopropyl alcohol according to claim 1,
wherein a reducing agent used in the step (3) includes hydrogen obtained in the step (2), and
a percentage of an amount of carbon dioxide in all components introduced in the step (3) is less than 10 mol %.

6. The method for producing isopropyl alcohol according to claim 1,
wherein the ethanol is derived from biomass as a raw material.

7. The method for producing isopropyl alcohol according to claim 6, the method comprising:
the step (1) of reacting ethanol and water in the presence of a catalyst to obtain acetone;
the step (2) of purifying the acetone; and
the step (3) of reducing the acetone to obtain isopropyl alcohol, wherein a reducing agent used in the step (3) includes hydrogen obtained in the step (2), and
a percentage of an amount of carbon dioxide in all components introduced in the step (3) is less than 10 mol %.

8. The method for producing isopropyl alcohol according to claim 1,
wherein the step (3) of reducing the acetone to obtain isopropyl alcohol is performed in the presence of a catalyst in which a metal element is supported on a support selected from activated carbon, silica, alumina, titania, zirconia, ceria, magnesia, and diatomaceous earth.

9. The method for producing isopropyl alcohol according to claim 1,
wherein a reducing agent used in the step (3) includes hydrogen obtained in the step (2),
a percentage of an amount of carbon dioxide in all components introduced in the step (3) is less than 10 mol %, and
the step (3) of reducing the acetone to obtain isopropyl alcohol is performed in the presence of a catalyst in which a metal element is supported on a support selected from activated carbon, silica, alumina, titania, zirconia, ceria, magnesia, and diatomaceous earth.

10. The method for producing isopropyl alcohol according to claim 1,
wherein the step (1) of reacting ethanol and water in the presence of a catalyst to obtain acetone is performed in the presence of a catalyst containing one or more elements selected from alkali metals, alkaline earth metals, iron, manganese, zinc, copper, aluminum, and zirconium.

11. The method for producing isopropyl alcohol according to claim 1,
wherein the step (1) of reacting ethanol and water in the presence of a catalyst to obtain acetone is performed in the presence of a catalyst containing iron and zirconium, the catalyst containing the zirconium in an amount of 0.2 to 2.0 mol per mole of the iron.

12. The method for producing isopropyl alcohol according to claim 1,
wherein a catalyst used in the step (3) includes a Ru-containing catalyst.

13. The method for producing isopropyl alcohol according to claim 1,
the method further comprising a step (4) of purifying hydrogen contained in the gas phase component obtained in the step (2),
wherein a molar ratio of hydrogen/carbon dioxide in a hydrogen-rich composition obtained in the step (4) is 90:10 or more.
